(19) United States
(12) Patent Application Publication
(10) Pub. No.: US 2014/0031691 A1
(43) Pub. Date: Jan. 30, 2014

(54) ULTRASOUND DIAGNOSTIC DEVICE

(71) Applicants: Yuko Nagase, Mitaka-shi (JP); Noriyoshi Matsushita, Mitaka-shi (JP)

(72) Inventors: Yuko Nagase, Mitaka-shi (JP); Noriyoshi Matsushita, Mitaka-shi (JP)

(73) Assignee: HITACHI ALOKA MEDICAL, LTD., Mitaka-shi, Tokyo (JP)

(21) Appl. No.: 14/111,129

(22) PCT Filed: Apr. 5, 2012
(86) PCT No.: PCT/JP2012/059346

§ 371 Date: Oct. 10, 2013

(30) Foreign Application Priority Data
Apr. 14, 2011 (JP) 2011-089927

(51) Int. Cl. A61B 8/08; A61B 8/13

(52) U.S. Cl. CPC A61B 8/5207; A61B 8/13
USPC 600/443

(57) ABSTRACT

A diagnostic coordinate system has first through third axes based on a major axis and a minor axis of a follicle, the origin being the position of the center of gravity of the follicle. The diagnostic coordinate system is translated with respect to an XYZ display coordinate system, rotating the diagnostic coordinate system until the first axis corresponding to the major axis of the follicle overlaps the X-axis where the angle with the first axis is smallest, and rotating until the second axis corresponding to the minor axis of the follicle overlaps the Z-axis where the angle with the second axis is smallest. A tomographic image of the follicle is formed by an image in a plane that includes the Z and X axes, an image in a plane that includes the Y and Z axes, and an image in a plane that includes the X and Y axes.

### TECHNICAL FIELD

The present invention relates to an ultrasound diagnostic apparatus, and in particular to a technique for forming a display image of a diagnosis target.

### BACKGROUND ART

Techniques for displaying an ultrasound image of a tissue or the like in a display image suited for diagnosis are known from the related art, and various display images exist as the display image for these techniques, corresponding to the type of the tissue or the like and the contents of the diagnosis. As the tissue or the like, a plurality of follicles in a living body are in some cases a target of ultrasound diagnosis.

Each follicle is in many cases observed in a shape approximately close to an ellipse, and, for example, a major axis of each follicle along a longitudinal direction thereof and minor axes orthogonal to the major axis are used as measurements in diagnosis of each follicle. Because of this, for example, many users desire a cutting-plane image including the major axis and the minor axes of the follicle when the ultrasound image data is three-dimensionally obtained and the cutting-plane image of each follicle is displayed.

However, in the process of setting the position of the cutting plane for the follicle to include the major axis and the minor axes of the follicle, if the setting is to be manually performed by the user, the user is forced to execute complicated operation. In particular, in a living body, a large number of follicles exist close to each other at high density, and the manual setting of the cutting plane for each of the large number of follicles requires a great amount of work.

In such a circumstance, the present inventors have researched and developed techniques for forming a display image suitable for diagnosis for a diagnosis target such as, for example, the follicle. In the formation of the display image, it is desirable to refer to the form of the diagnosis target; that is, the size and shape of the diagnosis target. An example of a feature quantity showing the form of the diagnosis target is the major axis identified along a longitudinal direction of the diagnosis target (refer to Patent Document 1)

### RELATED ART REFERENCES

### Patent Document

[Patent Document 1] Japanese Patent No. 3802508

### DISCLOSURE OF INVENTION

### Technical Problem

The present invention was conceived in the process of the above-described research and development, and an advantage thereof is realization of a display image of a diagnosis target according to the form of the diagnosis target.

### Solution to Problem

According to one aspect of the present invention, there is provided an ultrasound diagnostic apparatus comprising a probe which transmits and receives ultrasound to and from a diagnostic region; a transmitting and receiving unit which controls the probe to obtain a reception signal from the diagnostic region; a target identifying unit which identifies image data of a diagnosis target in image data of the diagnostic region formed based on the reception signal; a coordinate system setting unit which sets, based on the image data of the diagnosis target, a diagnostic coordinate system based on a form of the diagnosis target; a coordinate system matching unit which matches with each other a display coordinate system forming a basis of a display image and the diagnostic coordinate system, to place the image data of the diagnosis target in the display coordinate system; and a display image forming unit which forms a display image of the diagnosis target based on the image data of the diagnosis target placed in the display coordinate system.

In the above-described configuration, the image data of the diagnostic region may be formed, for example, with a plurality of echo data which are two-dimensionally arranged or a plurality of voxel data which are three-dimensionally arranged. When the image data of the diagnostic region are two-dimensional data, the diagnostic coordinate system and the display coordinate system are desirably two-dimensional coordinates. When the image data of the diagnostic region are three-dimensional data, the diagnostic coordinate system and the display coordinate system are desirably three-dimensional coordinates or two-dimensional coordinates. In addition, although the diagnostic coordinate system and the display coordinate system are desirably orthogonal coordinate systems, there may be used coordinate systems other than the orthogonal coordinate system, such as coordinate systems suited for the form of scanning of the ultrasound probe.

In the above-described configuration, the display coordinate system and the diagnostic coordinate system are matched with each other. For example, one coordinate system is placed in the other coordinate system such that a certain matching condition is satisfied. The matching condition is, for example, a relative placement relationship between a coordinate axis and a coordinate plane defining one coordinate system, and a coordinate axis and a coordinate plane defining the other coordinate system, or the like. As an example, there exists a form in which one coordinate axis of the display coordinate system and one coordinate axis of the diagnostic coordinate system are overlapped with each other. Alternatively, one coordinate axis of the display coordinate system and one coordinate axis of the diagnostic coordinate system may be matched in a manner to intersect each other with a certain intersection angle.

With the above-described configuration, because the diagnostic coordinate system based on the form of the diagnosis target and the display coordinate system are matched with each other when the image data of the diagnosis target are to be placed in the display coordinate system, placement of the image data corresponding to the form of the diagnosis target is realized, and a display image of the diagnosis target according to the form of the diagnosis target can be formed.

According to another aspect of the present invention, there is provided an ultrasound image processor comprising a target identifying unit which identifies image data of a diagnosis target in ultrasound image data; a coordinate system setting unit which sets, based on the image data of the diagnosis target, a diagnostic coordinates system based on a form of the diagnosis target; a coordinate system matching unit which matches with each other the display coordinate system forming a basis of a display image and the diagnostic coordinate system, to place the image data of the diagnosis target in the display coordinate system; and a display image forming unit which forms a display image of the diagnosis target based on the image data of the diagnosis target placed in the display coordinate system.

According to another aspect of the present invention, for example, a program which realizes the functions of the target identifying unit, the coordinate system setting unit, and the coordinate system matching unit described above may be used to cause a computer to realize these functions so that the computer functions as the ultrasound image preprocessor described above.

### Advantageous Effects of invention

According to various aspects of the present invention, a display image of a diagnosis target according to the form of the diagnosis target can be formed.

### BRIEF DESCRIPTION OF DRAWINGS

FIG. 1 is a diagram showing an overall structure of an ultrasound diagnostic apparatus preferable for practicing the present invention.

FIG. 2 is a diagram for explaining a process performed in a target identifying unit.

FIG. 3 is a diagram for explaining scanning of a filter in a three-dimensional data space.

FIG. 4 is a diagram for explaining a filter process in a dilation process.

FIG. 5 is a diagram for explaining a major axis and two minor axes of a follicle.

FIG. 6 is a diagram showing a diagnostic coordinate system based on a follicle.

FIG. 7 is a diagram for explaining matching of a display coordinate system and a diagnostic coordinate system.

FIG. 8 is a diagram for explaining a cross section based on a display coordinate system.

FIG. 9 is a diagram showing a concrete example of a display image.

### BEST MODE FOR CARRYING OUT THE INVENTION

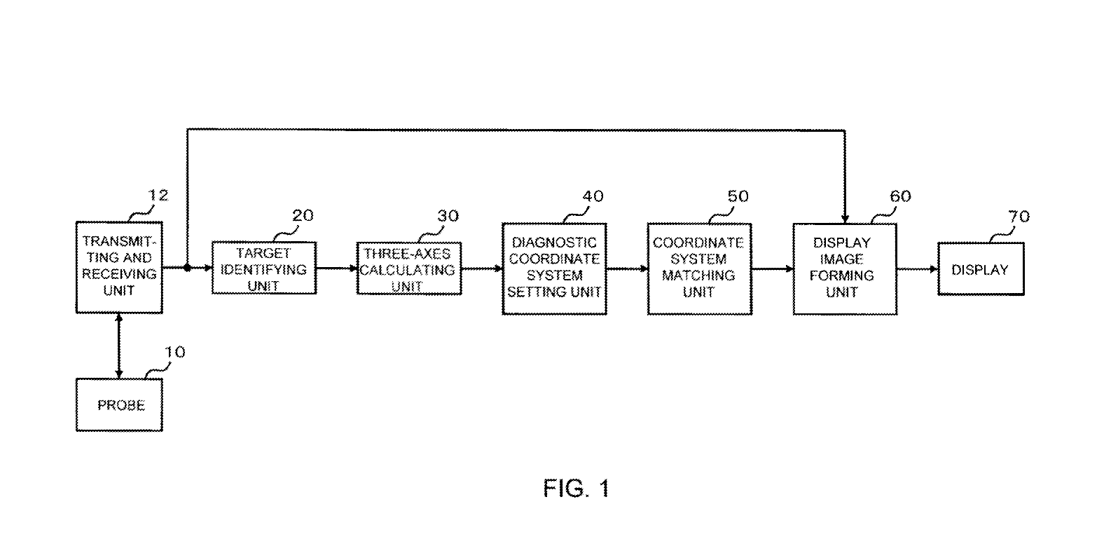

FIG. 1 is a diagram showing an overall structure of an ultrasound diagnostic apparatus preferable in practicing the present invention. A probe 10 is an ultrasound probe which transmits and receives ultrasound to and from a region including a diagnosis target. The probe 10 comprises a plurality of transducer elements which transmit and receive ultrasound. The plurality of transducer elements are transmission-controlled by a transmitting and receiving unit 12, to form a transmission beam. The plurality of transducer elements also receive ultrasound obtained from the region including a diagnosis target; a signal thus obtained is output to the transmitting and receiving unit 12; the transmitting and receiving unit 12 forms a reception beam; and echo data are collected along the reception beam.

As the probe 10, a three-dimensional probe which scans the ultrasound beam (transmission beam and reception beam) in a three-dimensional space and three-dimensionally collects the echo data is preferable. For example, a scanning plane electrically formed by a plurality of transducer elements which are arranged one-dimensionally (1-D array transducer) may be mechanically moved to three-dimensionally scan the ultrasound beam. Alternatively, a plurality of transducer elements arranged two-dimensionally (2-D array transducer) may be electrically controlled to three-dimensionally scan the ultrasound beam. Alternatively, there may be employed a two-dimensional ultrasound probe which scans the ultrasound beam within a tomographic plane.

When the ultrasound beam is scanned in the three-dimensional space and echo data are collected, echo data (voxel data) for a plurality of voxels forming the three-dimensional data space corresponding to the three-dimensional space are stored in a memory or the like (not shown). For the plurality of voxels forming the three-dimensional data space, various processes are executed by a target identifying unit 20 and the subsequent units. These processes will now be described. For a portion (structure) shown in FIG. 1, the reference numerals of FIG. 1 are used in the following description.

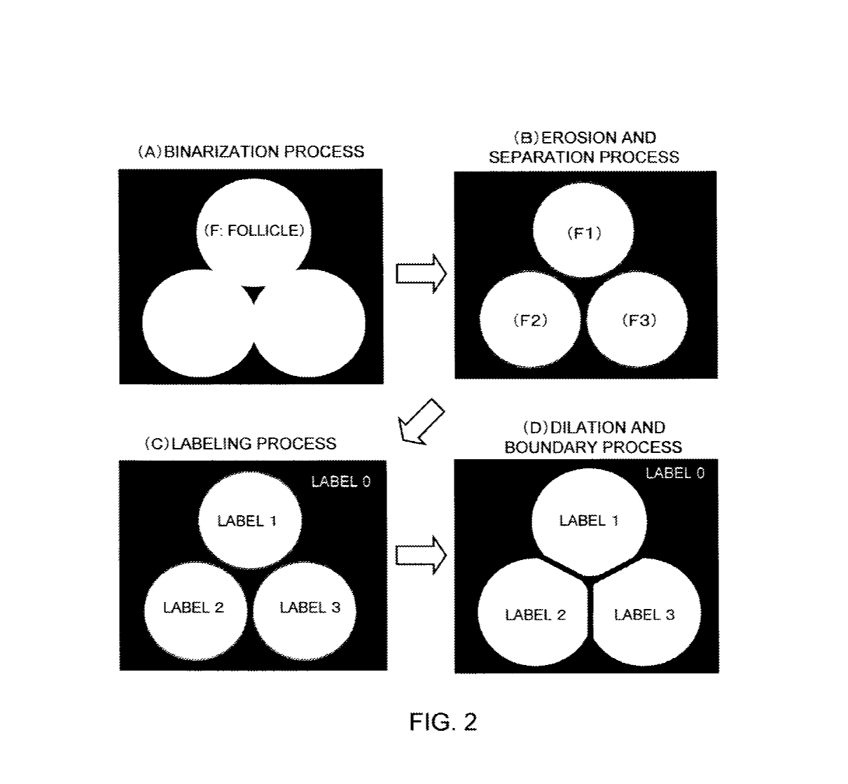

FIG. 2 is a diagram for explaining a process performed in the target identifying unit 20. FIG. 2(A) shows a binarization process. The target identifying unit 20 applies a binarization process on the plurality of voxels forming the three-dimensional data space, to form image data after the binarization process shown in FIG. 2(A). As the diagnosis target in the present embodiment, a follicle in a living body is preferable. The target identifying unit 20 compares the voxel value of each voxel (magnitude of echo data) with a threshold value for binarization, to distinguish voxels that correspond to the follicle F and voxels that do not. Then, for example, a voxel value of a voxel corresponding to the follicle F is set to “1” and a voxel value of the other voxels is set to “0.” In FIG. 2(A), a group of voxels corresponding to the follicle F is shown by a white color and a group of the other voxels corresponding to the background is shown by a black color.

In a living body, the plurality of follicles exist at high density, very close to each other. Therefore, in the ultrasound image, as shown in FIG. 2(A), an image of the plurality of follicles F is formed such that the follicles F are connected to each other, and it is difficult to individually check the size, shape, etc., of each follicle F. In consideration of this, in the present embodiment, the plurality of follicles F are separated into individual follicles by various processes to be described below. In FIG. 2, although each set of image data is two-dimensionally drawn, the processes are three-dimensionally executed in the three-dimensional data space.

FIG. 2(B) shows an erosion and separation process. The target identifying unit 20 applies an erosion process on the plurality of follicles F in the voxel data forming the three-dimensional data space and to which the binarization process is applied; that is, in the binarized image data shown in FIG. 2(A), the plurality of follicles F are separated into follicles F1-F3, as shown in FIG. 2(B). The target identifying unit 20 repeatedly executes the erosion process to stepwise erode the follicle Fn times (where n is a natural number). For the erosion process of each step, a filter for the erosion process is used, and the filter is scanned over the entire region of the three-dimensional data space.

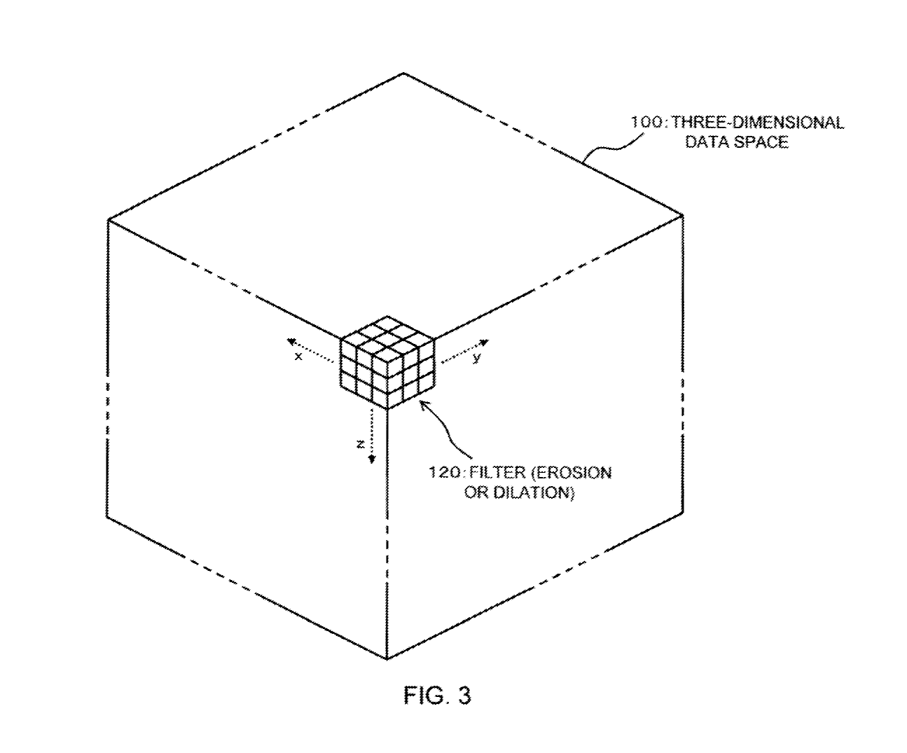

FIG. 3 is a diagram for explaining scanning of a filter 120 in three-dimensional data space 100. In FIG. 3, the three-dimensional data space 100 is shown with an xyz orthogonal coordinate system. In addition, the filter 120 has a three-dimensional structure with lengths in the x-axis direction, the y-axis direction, and z-axis direction each corresponding to three voxels, and, consequently, with a volume corresponding to a total of 27 voxels. A voxel positioned at the center of the filter 120 is a voxel of interest, and 26 voxels surrounding the voxel of interest are peripheral voxels. The filter 120 is scanned over the entire region of the three-dimensional data space 100 by being moved in the x-axis direction, the y-axis direction, and the z-axis direction, so that each of the voxels in the three-dimensional data space 100 is set as the voxel of interest.

In the erosion and separation process, at each scan position, if there is at least one voxel with the voxel value of “0” among the 26 peripheral voxels in the filter 120, the voxel value of the voxel of interest positioned at the center of the filter 120 is set as “0.” For example, when the voxel of interest has a voxel value of “1” (follicle), and at least one of the peripheral voxels has a voxel value of “0” (background), the voxel value of the voxel of interest is converted to “0” (background). By the filter 120 being scanned once over the entire region of the three-dimensional data space 100 and the filtering process being executed for each scan position, the erosion process of one step is completed. The conversion of the voxel value with regard to the voxel of interest is executed after the filter 120 is scanned once over the entire region of the three-dimensional data space 100. In other words, the conversion of voxel value is not executed in the middle of scanning of the filter 120, and the filter process is executed at any scan position based on the voxel value before the conversion.

When the erosion process of one step is completed as described above and the voxel value is converted based on the result of the erosion process, an erosion process of a second step is executed on the three-dimensional data space 100 formed of the converted voxel values. In the erosion process of second step also, the same filter process as the erosion process of the first step is executed. Specifically, in each scan position, if there is at least one voxel with a voxel value of “0” among the 26 peripheral voxels in the filter 120, the voxel value of the voxel of interest positioned at the center of the filter 120 is converted to “0.” The conversion of the voxel value is executed after the filter 120 is once scanned over the entire region of the three-dimensional data space 100.

The target identifying unit 20 repeatedly executes the stepwise erosion process n times (where n is a natural number). The number of repetitions n is suitably determined according to the size of each voxel, the size of the filter, etc., and is set, for example, to be about 10 or less. Alternatively, there may be employed a configuration in which the user can adjust the number n.

In the case of two-dimensional configuration, in place of the filter 120 shown in FIG. 3, a two-dimensional filter having the length and width corresponding to 3 voxels, and consequently, an area of a total of 9 voxels, may be used, a vowel positioned at the center may be set as the voxel of interest, and the 8 voxels surrounding the voxel of interest may be set as the peripheral voxel.

Referring again to FIG. 2, when the data are separated as a result of the erosion process into a plurality of follicles F1-F3 as shown in FIG. 2(B), the target identifying unit 20 applies a labeling process in the vowel data forming the three-dimensional data space and to which the erosion process is applied; that is, in the image data after the erosion process shown in FIG. 2(B), different labels are assigned to the plurality of follicles F1-F3. As the labeling process, known methods may be employed. For example, a block of a plurality of voxels having the same voxel value in the three-dimensional data space is detected, and a label number is assigned for each block. For example, as shown in FIG. 2(C), a label of 0 is assigned to the background portion which is a block with the voxel value of “0,” and labels of 1-3 are assigned to the follicles F1-F3, respectively, which are blocks with the voxel value of “1.”

After the labeling process is applied, the target identifying unit 20 applies a dilation process on each of a plurality of follicles in the voxel data forming the three-dimensional data space and to which the labeling process is applied; that is, the image data after the labeling process shown in FIG. 2(C). In a dilation portion obtained from each follicle in the dilation process, the label of the follicle is assigned, and the sizes of the plurality of follicles are restored while a boundary is formed at an overlap portion of the dilation portions (dilated follicle) which overlap each other due to the dilation process. With this process, as shown in FIG. 2(D), while the boundary (background pixel) is formed between the follicles corresponding to labels different from each other, the sizes of the follicles are restored to the sizes before the erosion process (immediately after the binarization process).

The target identifying unit 20 repeatedly executes the dilation process to stepwise dilate the follicle F n times (where n is the same number as the number of erosion processes). In the dilation process at each step, a filter for the dilation process is used, and the filter is scanned over the entire region in the three-dimensional data space. In the dilation process also, the three-dimensional filter 120 corresponding to a total of 27 voxels shown in FIG. 3 is used, a vowel positioned at the center of the filter 120 is set as the voxel of interest, and the 26 voxels surrounding the voxel of interest are set as the peripheral voxels. The filter 120 is moved in the x-axis direction, the y-axis direction, and the z-axis direction and scanned over the entire region of the three-dimensional data space 100, so that each of the voxels in the three-dimensional data space 100 is set as the voxel of interest. However, the filter process in the dilation process differs from that in the erosion process.

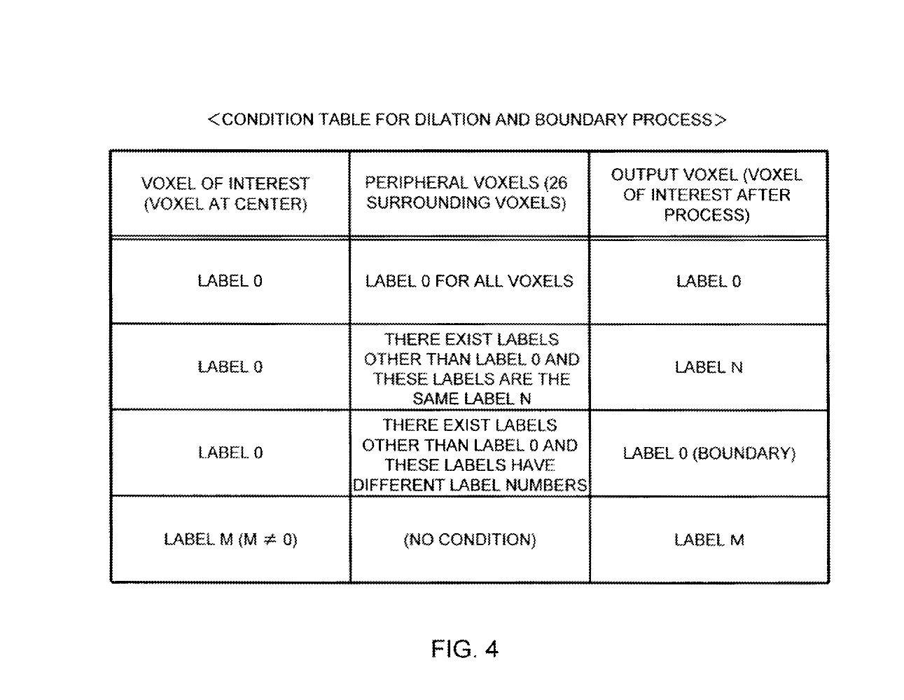

FIG. 4 is a diagram for explaining a filter process in the dilation process. FIG. 4 shows a condition table related to the conversion of the voxel value in the process to dilate while forming the boundary (dilation and boundary process). In the dilation and boundary process, reference is made to the label value of each voxel.

In the case where the voxel of interest positioned at the center of the filter 120 (FIG. 3) has a label of 0 (background), if all of 26 peripheral voxels have the label of 0 (background), the voxel of interest is set to a label of 0. In the case where the voxel of interest has the label of 0 (background), if there is a label (follicle) other than the label of 0 among the 26 peripheral voxels and all of the labels are the same label of N (same follicle), the voxel of interest is converted to the label of N. In other words, the follicle of the label of N is dilated.

In the case when the voxel of interest has a label of 0 (background), if there are labels (follicle) other than the label of 0 among the 26 peripheral voxels and the labels include different label numbers (follicles different from each other), the voxel of interest is set to the label of 0. In other words, the voxel of interest is maintained at the label of 0, and becomes a boundary between follicles which differ from each other.

On the other hand, when the voxel of interest positioned at the center of the filter 120 has a label of M (follicle), the voxel of interest is maintained with the label of M regardless of the status of the peripheral voxels.

When the filter 120 shown in FIG. 3 is scanned once over the entire region of the three-dimensional data space 100 and the filter process is applied at each scan position according to the condition shown in FIG. 4, a dilation and boundary process of one step is completed. The conversion of the label value of the voxel of interest is executed after the filter 120 is scanned once over the entire region of the three-dimensional data space 100. In other words, in the middle of scanning of the filter 120, the conversion of the label value is not executed, and the filter process is executed for the label value before the conversion for all scan positions.

When the dilation and boundary process of one step is completed in this manner and the label value is converted based on the result, the dilation and boundary process of a second step is executed on the three-dimensional data space 100 formed of the converted label values. In the dilation and boundary process of the second step also, the filter process identical to that of the first step is executed. Specifically, at each scan position, the filter process is executed according to the condition shown in FIG. 4, and, after the filter 120 is scanned once over the entire region of the three-dimensional data space 100, the label value is converted.

The target identifying unit 20 repeatedly executes the stepwise dilation and boundary process n times. The number of repetitions n is desirably identical to the number of repetitions n of the erosion process. In this manner, as shown in FIG. 2(D), while a boundary (background pixel) is formed between follicles corresponding to labels different from each other, the size of each follicle is restored to the size before the erosion process.

In the dilation and boundary process also, when a two-dimensional configuration is employed, in place of the filter 120 shown in FIG. 3, a two-dimensional filter corresponding to a length and a width of 3 voxels and a total of 9 voxels may be used, a voxel positioned at the center may be set as the voxel of interest, and the 8 voxels surrounding the voxel of interest may be set as the peripheral voxels.

In the present embodiment, as shown in FIG. 2, the plurality of follicles which exist at high density and close to each other are separated from each other and identified. In addition, as shown in FIG. 2(D), when an individual label is correlated to each follicle, corresponding follicles can be identified with the labels, and, for each label, calculation or the like of the measurement values related to the size and shape of each follicle corresponding to the label can be enabled. For example, a volume, a length of the major axis, a length of a minor axis, or the like of each follicle corresponding to each label may be calculated for each label.

In the process of identifying each follicle, for example, the user can designate a desired label to identify the follicle corresponding to the label. In addition, because the follicles are separated, on an image displaying the plurality of follicles, the user may designate a desired follicle by operating a display form such as a cursor, so that only an image of the follicle thus designated is displayed.

In the present embodiment, when the follicle designated by the user is displayed, a display image corresponding to the form of the follicle is formed. As a feature quantity related to the form of the follicle, a three-axes calculating unit 30 shown in FIG. 1 identifies a major axis and two minor axes of the follicle.

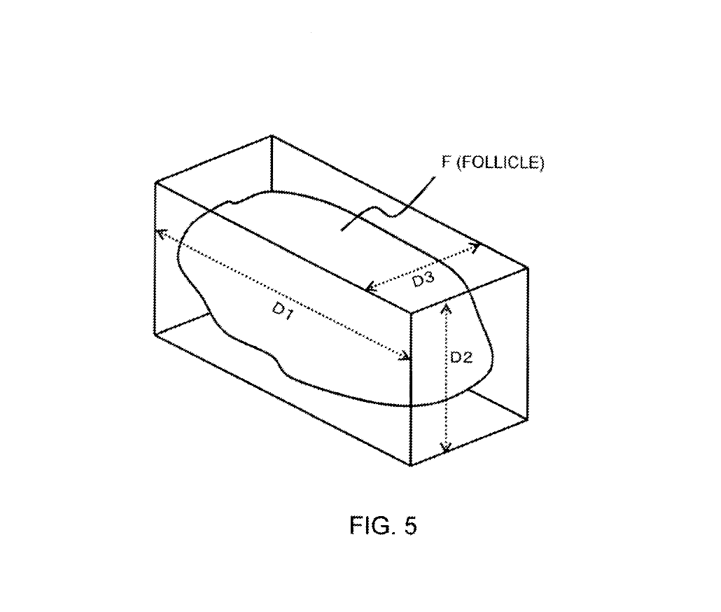

FIG. 5 is a diagram for explaining a major axis and two minor axes of the follicle. In the present embodiment, for the identified follicle F, as shown in FIG. 5, a minimum rectangular parallelepiped circumscribing the follicle F is considered, and lengths of the sides of the rectangular parallelepiped are set as the three axial lengths of the follicle F. For example, the longest side D1 shown in FIG. 5 is set as the major axis of the follicle F, and the sides D2 and D3 orthogonal to the side D1 are set as two minor axes of the follicle F.

Referring again to FIG. 1, the three-axes calculating unit 30 uses a method of primary component analysis in order to identify the three axes of the follicle. In the known method of primary component analysis, a direction which most represents the variation of the data; that is, a direction having the maximum variance of the data, is set as a first primary component. In the present embodiment, in the primary component analysis, for example, the following known covariance matrix is used.

In order to obtain a covariance matrix, an average position m is calculated by Equation 1. In Equation 1, Pᵢ represents a coordinate value in the three-dimensional data space (refer to FIG. 3) for an ith pixel (voxel) forming the follicle, and the average position (position of the center of gravity) m is calculated using the coordinate values of all pixels (voxels) of i=1−N forming the identified follicle.

$$m = \frac{1}{N} {\sum }_{i = 1}^{N} {P}_{i} , \\ {P}_{i} = 〈 {x}_{i} , {y}_{i} , {z}_{i} 〉 \qquad [ Equation 1 ]$$

Using the average position m of Equation 1, a covariance matrix C shown in Equation 2 is calculated. The covariance matrix C shown in Equation 2 is a 3×3 matrix, and is a symmetric matrix having 6 independent components shown in Equation 3.

$$\begin{matrix}m = \frac{1}{N} {\sum }_{i = 1}^{N} {P}_{i} , \\ {P}_{i} = 〈 {x}_{i} , {y}_{i} , {z}_{i} 〉 & [ Equation 2 ] \\ {C}_{11} = \frac{1}{N} {\sum }_{i = 1}^{N} {( {x}_{i} - {m}_{x} )}^{2} \\ {C}_{12} = {C}_{21} = \frac{1}{N} {\sum }_{i = 1}^{N} ( {x}_{i} - {m}_{x} ) ( {y}_{i} - {m}_{y} ) \\ {C}_{22} = \frac{1}{N} {\sum }_{i = 1}^{N} {( {y}_{i} - {m}_{y} )}^{2} \\ {C}_{13} = {C}_{31} = \frac{1}{N} {\sum }_{i = 1}^{N} ( {x}_{i} - {m}_{x} ) ( {z}_{i} - {m}_{z} ) \\ {C}_{33} = \frac{1}{N} {\sum }_{i = 1}^{N} {( {z}_{i} - {m}_{z} )}^{2} \\ {C}_{23} = {C}_{32} = \frac{1}{N} {\sum }_{i = 1}^{N} ( {y}_{i} - {m}_{y} ) ( {z}_{i} - {m}_{z} ) & [ Equation 3 ]\end{matrix}$$

In the primary component analysis using the covariance matrix C, eigenvectors of the covariance matrix C obtained by Equations 2 and 3 are calculated, and an eigenvector corresponding to a maximum eigenvalue is set as the first primary component. In the present embodiment, a direction of the first primary component obtained using the covariance matrix C is set as the major axis of the follicle. With this process, the major axis passing through the center of gravity of the follicle and along the longitudinal direction of the follicle is identified. In addition, directions of a second primary component and a third primary component obtained using the covariance matrix C are set as the two minor axes of the follicle. For example, a direction of the second primary component is set as a first minor axis and a direction of the third primary component is set as a second minor axis. In this manner, the major axis and two minor axes orthogonal to the major axis are identified as three axes of the follicle.

Alternatively, in the image data of the follicle, the major axis may be set along a straight line connecting the center of gravity and a pixel which is farthest away from the center of gravity. However, because there may be a case where the farthest pixel is noise or the like, the setting of the major axis by the primary component analysis is more desirable.

When three axes are identified by the three-axes calculating unit 30, a diagnostic coordinate system setting unit 40 sets a diagnostic coordinate system based on the form of the follicle. The diagnostic coordinate system setting unit 40 sets a diagnostic coordinate system having three axes of the follicle as the coordinate axes.

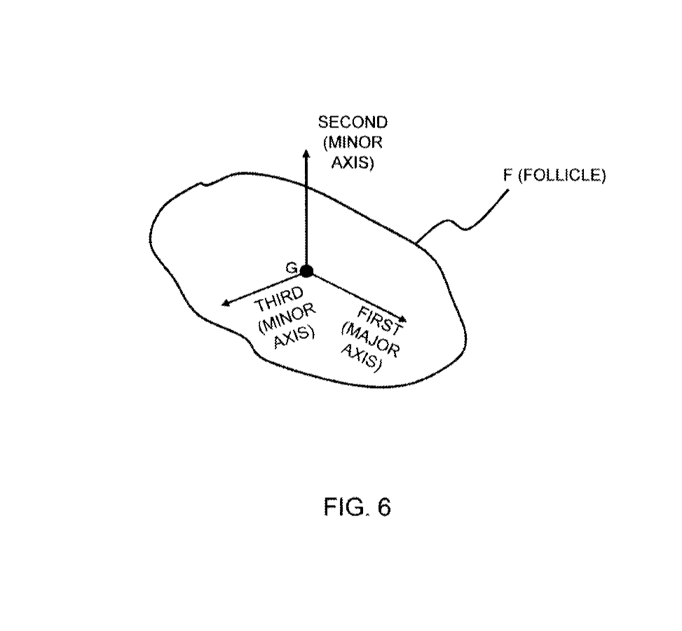

FIG. 6 is a diagram showing the diagnostic coordinate system based on the follicle. The diagnostic coordinate system setting unit 40 sets, as the diagnostic coordinate system, an orthogonal coordinate system shown in FIG. 6 and having, as an origin of the coordinates, a position of the center of gravity G of the follicle F, and having, as the coordinate axes, a first axis in the direction of the first primary component; that is, the direction of the major axis of the follicle F, a second axis in the direction of the second primary component; that is, the direction of one minor axis of the follicle F, and a third axis in the direction of the third primary component; that is, the direction of the other minor axis of the follicle F.

Referring again to FIG. 1, when the diagnostic coordinate system is set by the diagnostic coordinate system setting unit 40, a coordinate system matching unit 50 matches with each other a display coordinate system forming a basis of the display image and the diagnostic coordinate system, to place the image data of the follicle in the display coordinate system.

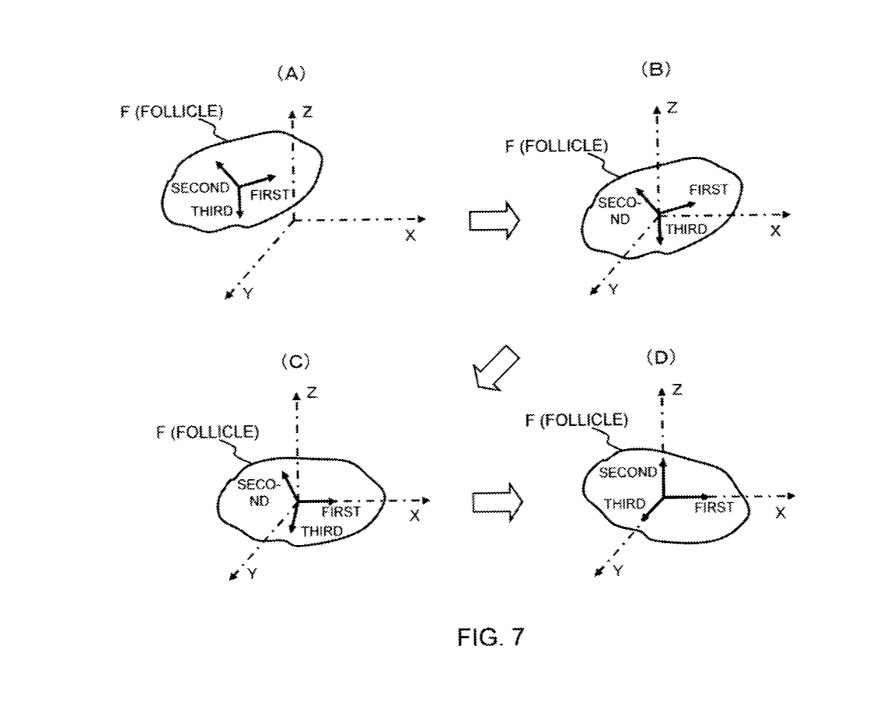

FIG. 7 is a diagram for explaining the matching of the display coordinate system and the diagnostic coordinate system. In FIG. 7, the display coordinate system is shown as an XYZ orthogonal coordinate system. The display coordinate system is a coordinate system which forms a basis when the display image is formed, and is a coordinate system having a clear relative position relationship with respect to the three-dimensional data space (refer to FIG. 3). In the present embodiment, the XYZ orthogonal coordinate system of the three-dimensional data space (refer to FIG. 3) is set as the display coordinate system without further processing.

In FIG. 7, the diagnostic coordinate system is a coordinate system identified by the first axis, second axis, and third axis (refer to FIG. 6). The first through third axes of the diagnostic coordinate system are axes which are obtained by the primary component analysis using, for example, Equations 1-3, based on the coordinates of the pixels (voxels) in the three-dimensional data space, and the position and direction in the three-dimensional data space are identified. Therefore, when the XYZ orthogonal coordinate system of the three-dimensional data space is set as the display coordinate system, the position and direction of the diagnostic coordinate system with respect to the display coordinate system are identified.

FIG. 7 shows in (A) an example of the diagnostic coordinate system (first through third axes) with respect to the follicle F identified on the display coordinate system (XYZ axes). Because the diagnostic coordinate system is a coordinate system based on the major axis and the minor axes of the follicle F, the diagnostic coordinate system corresponds to the position and orientation of the follicle F in the display coordinate system.

In consideration of the above, the coordinate system matching unit 50 first translates the diagnostic coordinate system with respect to the display coordinate system to coincide the origin of the display coordinate system and the origin of the diagnostic coordinate system. In this process, the voxel data (image data) related to the follicle F is also translated with the diagnostic coordinate system.

FIG. 7 shows in (B) a state where the diagnostic coordinate system is translated. The origin of the diagnostic coordinate system is moved to the position of the origin of the display coordinate system, and, with this process, the position of the center of gravity of the follicle F which is the origin of the diagnostic coordinate system is moved to the origin of the display coordinate system.

Then, the coordinate system matching unit 50 compares the axis corresponding to the major axis of the follicle F; that is, the first axis of the diagnostic coordinate system, and each of the XYZ axes of the display coordinate system, and identifies, among the XYZ axes, an axis having a smallest angle with respect to the first axis. For example, inner products between the first axis and the XYZ axes are compared to identify the axis having the smallest angle with respect to the first axis. The diagnostic coordinate system is then rotationally moved such that the identified axis and the first axis overlap each other. For example, when the X-axis is identified as the axis having the smallest angle with respect to the first axis, as shown in (C) of FIG. 7, the diagnostic coordinate system is rotated so that the first axis overlaps the X-axis, and the image data of the follicle F are also rotated.

The coordinate system matching unit 50 then compares the second axis of the diagnostic coordinate system corresponding to the minor axis of the follicle F and the remaining axes of the display coordinate system, and identifies an axis having a smallest angle with respect to the second axis. For example, when the first axis and the X axis are overlapped, among the remaining axes; that is, the Y-axis and the Z axis, the axis having the smallest angle with respect to the second axis is identified. The diagnostic coordinate system is then rotationally moved so that the identified axis and the second axis overlap each other. For example, when the Z axis is identified as having the smallest angle with respect to the second axis, as shown in (D) of FIG. 7, the diagnostic coordinate system is rotated such that the second axis overlaps the Z axis, and the image data of the follicle F are also rotated.

When the diagnostic coordinate system is an orthogonal coordinate system, if the first axis and the second axis are overlapped with the X axis and the Z axis, the third axis is placed along the Y axis. In (D) of FIG. 7, the third axis and the Y axis are overlapped in the same direction from each other.

Referring again to FIG. 1, when the display coordinate system and the diagnostic coordinate system are matched by the coordinate system matching unit 50 and the image data of the follicle are placed in the display coordinate system, a display image forming unit 60 forms a display image of the follicle based on the image data of the follicle placed in the display coordinate system, and the formed display image is displayed on a display 70. In the formation of the display image, a tomographic image of the follicle in a cross section based on the display coordinate system is formed.

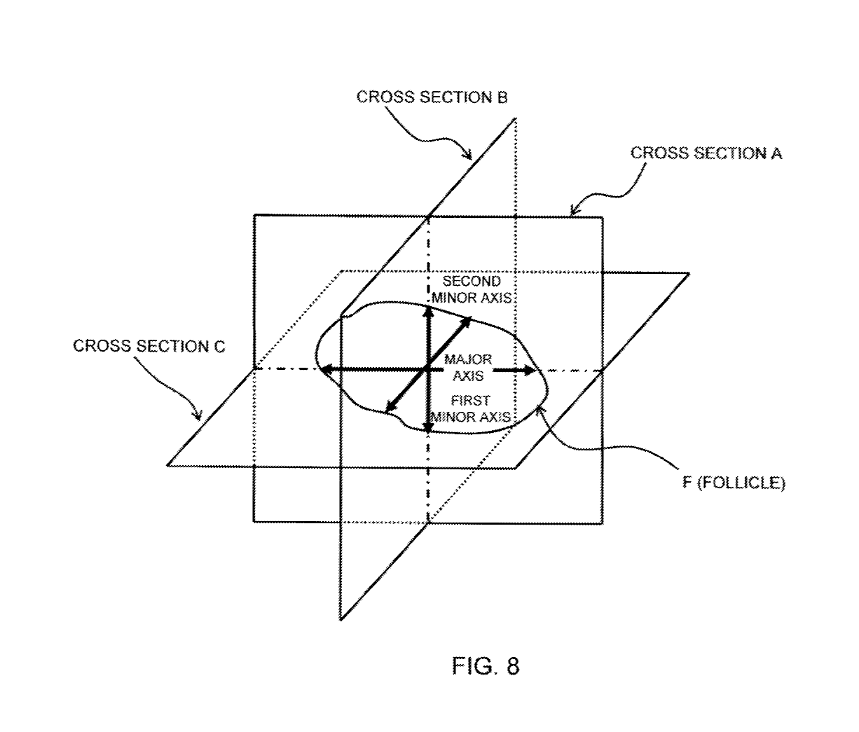

FIG. 8 is a diagram for explaining a cross section based on the display coordinate system. FIG. 8 shows the image data of the follicle F placed in the display coordinate system by the matching of the display coordinate system and the diagnostic coordinate system shown in FIG. 7(D). In FIG. 8, a cross section A is a plane including the Z axis and the X axis of the display coordinate system, a cross section B is a plane including the Y axis and the Z axis of the display coordinate system, and a cross section C is a plane including the X axis and the Y axis of the display coordinate system.

Because the display coordinate system and the diagnostic coordinate system are matched as shown in FIG. 7(D), the major axis of the follicle F corresponding to the first axis is placed on the X axis, the first minor axis of the follicle F corresponding to the second axis is placed on the Z axis, and the second minor axis of the follicle F corresponding to the third axis is placed on the Y axis. Therefore, in FIG. 8, the cross section A is a cross section including the major axis and the first minor axis of the follicle F, the cross section B is a cross section including the first minor axis and the second minor axis of the follicle F, and the cross section C is a cross section including the major axis and the second minor axis of the follicle F.

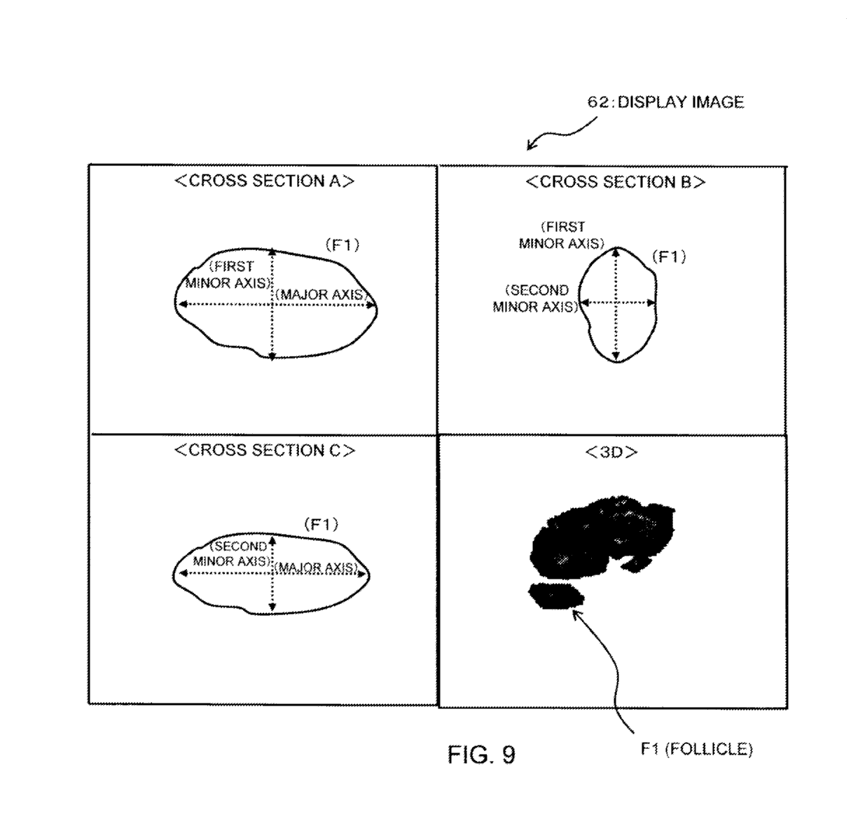

FIG. 9 is a diagram showing a concrete example of a display image 62. Of the images forming the display image 62 shown in FIG. 9, <3D> indicates a three-dimensional image related to a plurality of follicles. The three-dimensional image is formed by, for example, a volume rendering process based on the echo data (voxel data) collected from within the three-dimensional space. By employing a configuration where a position of a viewpoint in the volume rendering process can be changed by the user, it is possible to obtain a three-dimensional image displaying the plurality of follicles from a desired direction.

For example, in the three-dimensional image shown in FIG. 9, when the user displays the plurality of follicles from a desired direction, and moves a display form such as the cursor to position the display form on the position of the desired follicle, the user can select a follicle which the user wishes to diagnose. With this process, a follicle F1 is identified in the three-dimensional image of FIG. 9. Alternatively, in the three-dimensional image, with a display form such as colors and markers or the like, the display may be formed to allow visual distinction of the identified follicle from the other follicles.

For example, when the user identifies the follicle F1 using the three-dimensional image, the three-axes calculating unit 30 identifies the three axes of the follicle F1 (refer to FIG. 5 and Equations 1-3), the diagnostic coordinate system setting unit 40 sets the diagnostic coordinate system corresponding to the three axes of the follicle F1 (refer to FIG. 6), and the coordinate system matching unit 50 matches the display coordinate system and the diagnostic coordinate system (refer to FIG. 7). The display image forming unit 60 then forms tomographic images of the follicle F1 at the cross sections A-C (refer to FIG. 8).

In FIG. 9, of the images forming the display image 62, the <cross section A> indicates a tomographic image on the cross section A of the follicle F1, the <cross section B> indicates a tomographic image on the cross section B of the follicle F1, and the <cross section C> indicates a tomographic image on the cross section C of the follicle F1. Because the display coordinate system and the diagnostic coordinate system are matched, on the cross section A, a cross section including the major axis and the first minor axis of the follicle F1 is displayed, on the cross scion B, a cross section including the first minor axis and the second minor axis of the follicle F1 is displayed, and on the cross section C, a cross section including the major axis and the second minor axis of the follicle F1 is displayed.

In this manner, in the present embodiment, the user selects a desired follicle from a plurality of follicles, and a tomographic image including three axes of the identified follicle is formed. Because of this, complicated operation by the user, for example, an operation for setting the cutting plane or the like, can be reduced, and, desirably, the operation for setting the cutting plane can be omitted.

In addition, in the matching of the display coordinate system and the diagnostic coordinate system, the coordinate axes having the minimum intersecting angle are overlapped, and, thus, the rotational movement of the diagnostic coordinate system can be minimized, and visual discomfort of the user felt due to the rotational movement can be minimized.

Alternatively, for the identified follicle F1, measurement values such as the length of the major axis, the lengths of the two minor axes, and the volume may be displayed as a part of the display image 62. In addition, because the plurality of follicles are separated from each other and identified (refer to FIG. 2), the measurement values such as the length of the major axis, the lengths of two minor axes, and the volume for each follicle may be calculated, and a list of the measurement values for the plurality of follicles may be displayed. Moreover, the user may identify a desired follicle from the list of the measurement values, and a cross section of the follicle thus identified may be displayed.

An ultrasound diagnostic apparatus according to a preferred embodiment of the present invention has been described. Alternatively, for example, at least one of the target identifying unit 20, the three-axes calculating unit 30, the diagnostic coordinate system setting unit 40, the coordinate system matching unit 50, and the display image forming unit 60 shown in FIG. 1 may be realized by a computer, and the computer may function as the ultrasound image processor.

### EXPLANATION OF REFERENCE NUMERALS

10 PROBE; 20 TARGET IDENTIFYING UNIT; 30 THEE-AXES CALCULATING UNIT; 40 DIAGNOSTIC COORDINATE SYSTEM SETTING UNIT; 50 COORDINATE SYSTEM MATCHING UNIT; 60 DISPLAY IMAGE FORMING UNIT; 62 DISPLAY IMAGE 70 DISPLAY

The invention claimed is:

1. An ultrasound diagnostic apparatus comprising:
a probe which transmits and receives ultrasound to and from a diagnostic region;
a transmitting and receiving unit which controls the probe to obtain a reception signal from the diagnostic region;
a target identifying unit which identifies image data of a diagnosis target in image data of the diagnostic region formed based on the reception signal;
a coordinate system setting unit which sets, based on the image data of the diagnostic target, a diagnostic coordinate system based on a form of the diagnosis target;
a coordinate system matching unit which matches with each other a display coordinate system forming a basis of a display image and the diagnostic coordinate system, to place the image data of the diagnosis target in the display coordinate system; and
a display image forming unit which forms a display image of the diagnosis target based on the image data of the diagnosis target placed in the display coordinate system.

2. The ultrasound diagnostic apparatus according to claim 1, wherein
the coordinate system setting unit sets the diagnostic coordinate system including, as a coordinate axis, a reference axis identified according to the form of the diagnosis target, and
the coordinate system matching unit overlaps one coordinate axis of the display coordinate system and the reference axis with each other, to match the display coordinate system and the diagnostic coordinate system with each other.

3. The ultrasound diagnostic apparatus according to claim 2, wherein
the coordinate system setting unit sets the diagnostic coordinate system including, as a coordinate axis, a major axis identified along a longitudinal direction of the diagnosis target, and
the coordinate system matching unit overlaps one coordinate axis of the display coordinate system and the major axis with each other.

4. The ultrasound diagnostic apparatus according to claim 3, wherein
the coordinate system setting unit sets the diagnostic coordinate system including, as the coordinate axis and in addition to the major axis of the diagnosis target, a minor axis orthogonal to the major axis, and
the coordinate system matching unit overlaps one coordinate axis of the display coordinate system and the major axis with each other, and overlaps with each other another coordinate axis of the display coordinate system and the minor axis.

5. The ultrasound diagnostic apparatus according to claim 3, wherein
the coordinate system matching unit overlaps with each other a coordinate axis, of the plurality of coordinate axes of the display coordinate system, having a smallest angle with respect to the major axis, and the major axis.

6. The ultrasound diagnostic apparatus according to claim 4, wherein
the coordinate system matching unit overlaps with each other a coordinate axis, of the plurality of coordinate axes of the display coordinate system, having a smallest angle with respect to the major axis, and the major axis.

7. The ultrasound diagnostic apparatus according to claim 1, wherein
the coordinate system setting unit sets a diagnostic coordinate system obtained by a primary component analysis based on the image data of the diagnosis target.

8. The ultrasound diagnostic apparatus according to claim 3, wherein
the coordinate system setting unit sets a diagnostic coordinate system including, as a coordinate axis, a major axis of the diagnosis target obtained by a primary component analysis based on the image data of the diagnosis target.

9. The ultrasound diagnostic apparatus according to claim 4, wherein
the coordinate system setting unit sets a diagnostic coordinate system including, as coordinate axes, a major axis and a minor axis of the diagnosis target obtained by a primary component analysis based on the image data of the diagnosis target.

10. The ultrasound diagnostic apparatus according to claim 3, wherein
the coordinate system setting unit sets a diagnostic coordinate system including, as a coordinate axis, a major axis of the diagnosis target obtained by a primary component analysis based on the image data of the diagnosis target, and
the coordinate system matching unit overlaps with each other a coordinate axis, of the plurality of coordinate axes of the display coordinate system, having a smallest angle with respect to the major axis, and the major axis.

11. The ultrasound diagnostic apparatus according to claim 4, wherein
the coordinate system setting unit sets a diagnostic coordinate system including, as coordinate axes, a major axis and a minor axis of the diagnosis target obtained by a primary component analysis based on the image data of the diagnosis target, and
the coordinate system matching unit overlaps with each other a coordinate axis, of the plurality of coordinate axes of the display coordinate system, having a smallest angle with respect to the major axis, and the major axis.

12. The ultrasound diagnostic apparatus according to claim 1, wherein
the probe transmits and receives the ultrasound to and from the diagnostic region including a plurality of follicles which are in close contact to each other, and
in the image data of the diagnostic region including the plurality of follicles, the target identifying unit:
applies an erosion process on the plurality of follicles to separate the plurality of follicles into each individual follicle;
applies a labeling process on the plurality of follicles in the image data to which the erosion process is applied, to assign different labels to the plurality of follicles;
applies a dilation process on each of the plurality of the follicles in the image data to which the labeling process is applied, to restore sizes of the plurality of the follicles while assigning the label of each follicle to a dilation portion obtained from the follicle; and
identifies, in the image data, the image data of each follicle having the size restored as the image data of the diagnosis target.

13. The ultrasound diagnostic apparatus according to claim 12, wherein
the target identifying unit restores the sizes of the plurality of follicles while a boundary is formed in an overlap portion between dilation portions which overlap each other due to the dilation process.

14. The ultrasound diagnostic apparatus according to claim 12, wherein
the target identifying unit repeatedly executes an erosion process to stepwise erode the plurality of follicles n times (where n is a natural number), and repeatedly executes a dilation process to stepwise dilate the plurality of follicles n times.

15. An ultrasound image processor comprising:
a target identifying unit which identifies image data of a diagnosis target in ultrasound image data;
a coordinate system setting unit which sets, based on the image data of the diagnosis target, a diagnostic coordinate system based on a form of the diagnosis target;
a coordinate system matching unit which matches with each other a display coordinate system forming a basis of a display image and the diagnostic coordinate system, to place the image data of the diagnosis target in the display coordinate system; and
a display image forming unit which forms a display image of the diagnosis target based on the image data of the diagnosis target placed in the display coordinate system.
